SEQUENCE LISTING (9 sequences)

SEQ ID NO: 1 - DNA, 789 bp, Escherichia coli
  source 1..789: mol_type = genomic DNA
atgggttttctttccggtaagcgcattctggtaaccggtgttgccagcaaactatccatcgcctacggtatcgctcaggcgatgcaccgcgaaggagctgaactggcattcacctaccagaacgacaaactgaaaggccgcgtagaagaatttgccgctcaattgggttctgacatcgttctgcagtgcgatgttgcagaagatgccagcatcgacaccatgttcgctgaactggggaaagtttggccgaaatttgacggtttcgtacactctattggttttgcacctggcgatcagctggatggtgactatgttaacgccgttacccgtgaaggcttcaaaattgcccacgacatcagctcctacagcttcgttgcaatggcaaaagcttgccgctccatgctgaatccgggttctgccctgctgaccctttcctaccttggcgctgagcgcgctatcccgaactacaacgttatgggtctggcaaaagcgtctctggaagcgaacgtgcgctatatggcgaacgcgatgggtccggaaggtgtgcgtgttaacgccatctctgctggtccgatccgtactctggcggcctccggtatcaaagacttccgcaaaatgctggctcattgcgaagccgttaccccgattcgccgtaccgttactattgaagatgtgggtaactctgcggcattcctgtgctccgatctctctgccggtatctccggtgaagtggtccacgttgacggcggtttcagcattgctgcaatgaacgaactcgaactgaaataa

SEQ ID NO: 2 - DNA, 7920 bp, synthetic construct
  source 1..7920: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccgccaccatgtttcccatgcaattcacaaactcagcctatcgccagatggagcccatgttcgcaccgggtcctcgaggacaagtacagccgtaccggccgcgcactaagcgtcgccaagagccgcaagtcggcaacgctgctattgctgccctcgcgaaccagatgagcgcgctccagctacaggtggctggacttgccggccaggcaagggtggatcgtcgtggaccaagacgtgttcagaaaagcaagcagaagaagaagaatccctccaacggagaaaaacccaaggagaagaagaagaagcaaaaacaacaggagaagaaagggagcggtggcgtaatcaaaaaaccacggaaccggcccggaaaggaggtaaggatctccgtaaagcgtgcccgacagagcaccttccccgtgtaccatgacggtgctatatccggctacgctgtgctgattggttctcgcgtgtttaagccagcgcacgtgaagggtaagatcgaccaccccgagctggcggacatcaagttccaggtcgccgaggacatggacctcgaagcagctgcttaccctaagagtatgcgagaccaggcggctgaaccagcaaccatgatggacggagtgtataactgggagtacggcactatcagagtggaggacaatgtcgtaattgacgcgagcggcagaggtaagccgggtgacagtggaagggccattaccgacaactcaggaaaggttgttggcattgtcctcggagggggacccgacggcaggcgcacacgcctctccgtgataggtttcgacaagaagatgaaggctagagagatcgcctacagcgaggccataccttggacacgtgctccagccctcctgctgctgcccatggtcatcgcctgtacctacaactccaacacctttgattgctccaaaccgtcctgccaggactgttgtatcactgctgaaccagagaaggccatggctatgttgaaggacaacctgaacgacccgaactattgggacctactcattgccgtcaccacctgcaattccgcccggaaaaagagggctgtgtctgcgtcgcctgccgccgtttacgacacacaaatcctcgccgcccacgcagctgcctccccgtacagggcgtattgccccgactgtgatggaactgcctgcatctcgccgatagctattgacgaggtggtaagcagcggtagcgaccacgtcctccgcatccgggtcggttctcaatcgggagtgaccgctaaaggcggtgcggcgggtgaaacatctctgcgatacctgggaagggacggtaaggtgcacgccgcggacaacacgcggcttgtggtgcgcaccactgcaaagtgcgatgtgctgcaggccaccggccactacatcctggccagctgcccagaggggcagagtattactgttgcggccacactggacggcacccgccaccaatgcaccacggttttcgaacatcaagtaacggagaagttcaccagagaacgcagcaagggccaccacttgtccgacctgaccaagaagtgcaccagattttccaccaccccgaagaagtccgccccctacctcgttgacgtgtacgacgctctgccgatttccgtagagattagcaccgttgtaacatgcaacgacaatcagtgcacagtgagggtgtcacccggtaccacagtgaaattcgataagaagtgcaagagcgctgcccaagcgaccgttacctttaccagcgactcccagacgtttacgtgtgaggagccggttctgacggccgccagtatcacccagggcaagccgcaccttagatcatctatgttgcccagcggaggcaaggaagtgaaggcgaggatcccattcccgttcccgccagagaccgcgacctgcagagtaagtgtcgccccgctgccgtcgatcacctatgaggaaagcgacgttctgctggccggtaccgcgaagtaccccgtgctgctgactacacggaaccttggtttccacagcaatgccacatccgaatggatccagggtaagtacttgcgccgtatcccggtcacgccccaagggatcgaactaacgtggggaaataacgcaccgttgcacttctggtcatctgttaggtacgcatctggggacgccgacgcgtacccttgggaacttctggtgcaccacaccaagcaccatccggagtacgcgtgggcgtttgtaggagttgcatgtggtctgctggttattgcagtatgcatgttcgcgtgcgcatgcaacagagtgcggtactctttggtcgccaacacgttcaacccgaacccaccaccactgaccgcactgactgcagcattgtgctgcatacctggggctcgtgcggaccaaccctacctggacatcattgcctacctgtggaccaacagcaaagtggccttcgggctgcaatgcgcggcgcccgtggcttgcgtgctcatcgtcacatacgcccttagacactgcagactgtgctgcaagtcttttttaggggtaagagggtggtcagctctgctggtcatccttgcgtatgtacagagctgcaagagctacgaacacaccgtggtggtcccaatggacccgagagccccgtcgtacgaagcagtgataaaccggaatgggtatgatcccttgaagctgaccattgcagtgaactttaccgtcatctcaccaactacagctctggaatactggacttgcgcaagtgtccctatcgtcgagccgccccatgtgggctgctgcacgtcggtgtcctgcccctccgacctctccacactgcacgcgttcaccggcaaagccgtctccgacgtgcactgcgatgtgcacacaaacgtgtaccccttgttgtggggtgcggctcattgcttctgctccaccgagaatacacaagtcagcgcggtggctgccaccgtctctgaattttgtgctcaggactcagagcgcgccgaagcgtttagcgttcacagcagctcagtcactgcagaggttctggtgacgcttggtgaagtggtgacggcagtccacgtttacgtggacggggtaacatctgccaggggtaccgacctcaagatcgtggctgggccaataacaactgactactccccgttcgatcgcaaagtagtccgtatcggcgaagaggtctataactacgactggcctccttacggggctggtcgaccaggcacattcggggatattcaagctaggtcaaccaactatgtcaagcccaatgatctgtacggggatatcggaatcgaagtactgcagccgactaatgaccatgtgcacgtggcttacacgtatacgacctccgggttactgcgttggctgcaggacgctccgaagccacttagtgtcacagcaccgcacggttgtaagatcagtgccaacccgctcctggccctcgattgtggggttggcgccgtccccatgtccatcaacattccggacgcgaagttcacccgcaaattgaaggatccgaaaccatcggccctgaaatgcgtggtggacagttgcgagtacggggtggactacgggggcgccgccacgatcacctatgagggccacgaggctgggaagtgcgggatccattccctgacaccaggagtccctctgagaacatcagtggtcgaagtggttgccggcgctaacaccgtcaaaacgaccttctcctcacccacgcccgaggtcacactcgaggtagagatctgttcggcaatggtgacgtgcgccagtgagtgcactccaccgaaggaacatgtagtcgcaaccaggcctcgccatggcagcgacactggaggctacatctccgggcccgcaatgcgctgggccggtgggattgtagggaccttagcggtcctgttcctcatccttgccgttacctactgcgtggtgaagaagtgccgctctaaaagaatccggatagtcaagagctaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacatctagatgatcggcacgtaagaggttccaactttcaccataatgaaataagatcactaccgggcgtattttttgagttatcgagattttcaggagctaaggaagctaaaatgggttttctgagcggcaagcgtattctggtgaccggcgtggcgagcaaactgagcattgcgtatggtatcgcgcaagcgatgcaccgtgagggtgcggaactggcgttcacctaccagaacgataagctgaaaggtcgtgtggaggaatttgcggcgcagctgggtagcgacattgtgctgcaatgcgacgttgcggaggatgcgagcattgacaccatgttcgcggaactgggcaaagtgtggccgaaattcgatggctttgttcacagcatcggttttgcgccgggcgaccaactggacggtgattacgtgaacgcggttacccgtgagggcttcaagatcgcgcacgatattagcagctatagctttgttgcgatggcgaaagcgtgccgtagcatgctgaacccgggtagcgcgctgctgaccctgagctacctgggtgcggaacgtgcgattccgaactataacgtgatgggtctggcgaaggcgagcctggaggcgaacgttcgttatatggcgaacgcgatgggtccggaaggcgtgcgtgttaacgcgattagcgcgggtccgattcgtaccctggcggcgagcggcatcaaggacttccgtaaaatgctggcgcactgcgaggcggtgaccccgatccgtcgtaccgtgaccattgaagatgttggcaacagcgcggcgtttctgtgcagcgacctgagcgcgggtattagcggcgaggttgtgcatgttgatggcggctttagcattgcggcgatgaacgaactggaactgaaataaaccggtctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 3 - DNA, 8372 bp, synthetic construct
  source 1..8372: mol_type = other DNA
tcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaggctagagtacttaatacgactcactataggctagcctcgagaattcgccgccaccatgtttcccatgcaattcacaaactcagcctatcgccagatggagcccatgttcgcaccgggtcctcgaggacaagtacagccgtaccggccgcgcactaagcgtcgccaagagccgcaagtcggcaacgctgctattgctgccctcgcgaaccagatgagcgcgctccagctacaggtggctggacttgccggccaggcaagggtggatcgtcgtggaccaagacgtgttcagaaaagcaagcagaagaagaagaatccctccaacggagaaaaacccaaggagaagaagaagaagcaaaaacaacaggagaagaaagggagcggtggcgtaatcaaaaaaccacggaaccggcccggaaaggaggtaaggatctccgtaaagcgtgcccgacagagcaccttccccgtgtaccatgacggtgctatatccggctacgctgtgctgattggttctcgcgtgtttaagccagcgcacgtgaagggtaagatcgaccaccccgagctggcggacatcaagttccaggtcgccgaggacatggacctcgaagcagctgcttaccctaagagtatgcgagaccaggcggctgaaccagcaaccatgatggacggagtgtataactgggagtacggcactatcagagtggaggacaatgtcgtaattgacgcgagcggcagaggtaagccgggtgacagtggaagggccattaccgacaactcaggaaaggttgttggcattgtcctcggagggggacccgacggcaggcgcacacgcctctccgtgataggtttcgacaagaagatgaaggctagagagatcgcctacagcgaggccataccttggacacgtgctccagccctcctgctgctgcccatggtcatcgcctgtacctacaactccaacacctttgattgctccaaaccgtcctgccaggactgttgtatcactgctgaaccagagaaggccatggctatgttgaaggacaacctgaacgacccgaactattgggacctactcattgccgtcaccacctgcaattccgcccggaaaaagagggctgtgtctgcgtcgcctgccgccgtttacgacacacaaatcctcgccgcccacgcagctgcctccccgtacagggcgtattgccccgactgtgatggaactgcctgcatctcgccgatagctattgacgaggtggtaagcagcggtagcgaccacgtcctccgcatccgggtcggttctcaatcgggagtgaccgctaaaggcggtgcggcgggtgaaacatctctgcgatacctgggaagggacggtaaggtgcacgccgcggacaacacgcggcttgtggtgcgcaccactgcaaagtgcgatgtgctgcaggccaccggccactacatcctggccagctgcccagaggggcagagtattactgttgcggccacactggacggcacccgccaccaatgcaccacggttttcgaacatcaagtaacggagaagttcaccagagaacgcagcaagggccaccacttgtccgacctgaccaagaagtgcaccagattttccaccaccccgaagaagtccgccccctacctcgttgacgtgtacgacgctctgccgatttccgtagagattagcaccgttgtaacatgcaacgacaatcagtgcacagtgagggtgtcacccggtaccacagtgaaattcgataagaagtgcaagagcgctgcccaagcgaccgttacctttaccagcgactcccagacgtttacgtgtgaggagccggttctgacggccgccagtatcacccagggcaagccgcaccttagatcatctatgttgcccagcggaggcaaggaagtgaaggcgaggatcccattcccgttcccgccagagaccgcgacctgcagagtaagtgtcgccccgctgccgtcgatcacctatgaggaaagcgacgttctgctggccggtaccgcgaagtaccccgtgctgctgactacacggaaccttggtttccacagcaatgccacatccgaatggatccagggtaagtacttgcgccgtatcccggtcacgccccaagggatcgaactaacgtggggaaataacgcaccgttgcacttctggtcatctgttaggtacgcatctggggacgccgacgcgtacccttgggaacttctggtgcaccacaccaagcaccatccggagtacgcgtgggcgtttgtaggagttgcatgtggtctgctggttattgcagtatgcatgttcgcgtgcgcatgcaacagagtgcggtactctttggtcgccaacacgttcaacccgaacccaccaccactgaccgcactgactgcagcattgtgctgcatacctggggctcgtgcggaccaaccctacctggacatcattgcctacctgtggaccaacagcaaagtggccttcgggctgcaatgcgcggcgcccgtggcttgcgtgctcatcgtcacatacgcccttagacactgcagactgtgctgcaagtcttttttaggggtaagagggtggtcagctctgctggtcatccttgcgtatgtacagagctgcaagagctacgaacacaccgtggtggtcccaatggacccgagagccccgtcgtacgaagcagtgataaaccggaatgggtatgatcccttgaagctgaccattgcagtgaactttaccgtcatctcaccaactacagctctggaatactggacttgcgcaagtgtccctatcgtcgagccgccccatgtgggctgctgcacgtcggtgtcctgcccctccgacctctccacactgcacgcgttcaccggcaaagccgtctccgacgtgcactgcgatgtgcacacaaacgtgtaccccttgttgtggggtgcggctcattgcttctgctccaccgagaatacacaagtcagcgcggtggctgccaccgtctctgaattttgtgctcaggactcagagcgcgccgaagcgtttagcgttcacagcagctcagtcactgcagaggttctggtgacgcttggtgaagtggtgacggcagtccacgtttacgtggacggggtaacatctgccaggggtaccgacctcaagatcgtggctgggccaataacaactgactactccccgttcgatcgcaaagtagtccgtatcggcgaagaggtctataactacgactggcctccttacggggctggtcgaccaggcacattcggggatattcaagctaggtcaaccaactatgtcaagcccaatgatctgtacggggatatcggaatcgaagtactgcagccgactaatgaccatgtgcacgtggcttacacgtatacgacctccgggttactgcgttggctgcaggacgctccgaagccacttagtgtcacagcaccgcacggttgtaagatcagtgccaacccgctcctggccctcgattgtggggttggcgccgtccccatgtccatcaacattccggacgcgaagttcacccgcaaattgaaggatccgaaaccatcggccctgaaatgcgtggtggacagttgcgagtacggggtggactacgggggcgccgccacgatcacctatgagggccacgaggctgggaagtgcgggatccattccctgacaccaggagtccctctgagaacatcagtggtcgaagtggttgccggcgctaacaccgtcaaaacgaccttctcctcacccacgcccgaggtcacactcgaggtagagatctgttcggcaatggtgacgtgcgccagtgagtgcactccaccgaaggaacatgtagtcgcaaccaggcctcgccatggcagcgacactggaggctacatctccgggcccgcaatgcgctgggccggtgggattgtagggaccttagcggtcctgttcctcatccttgccgttacctactgcgtggtgaagaagtgccgctctaaaagaatccggatagtcaagagctaagcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataaggatccgggctggcgtaatagcgaagaggcccgcaccgatcgcccttcccaacagttgcgcagcctgaatggcgaatggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacatctagaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattattaagccgaaaatccgcggttttatctgcaccaccacccacccggttggttgcgaagcgaatgtgaaggaacaaattgcgtacaccaaggcgcagggcccgatcaagaacgcgccgaaacgtgttctggtggttggtagcagcagcggctatggtctgagcagccgtattgctgcggcgtttggtggcggtgcggcgaccattggcgtgttctttgagaaaccgggtaccgacaagaaaccgggcaccgcgggtttctacaacgctgcggcgtttgataagctggcgcacgaagcgggcctgtatgcgaaaagcctgaacggtgacgcgtttagcaacgaggcgaagcagaaagcgatcgaactgattaagcaggacctgggtcaaatcgatctggtggtttacagcctggcgagcccggttcgtaagatgccggataccggcgagctggtgcgtagcgcgctgaaaccgattggtgaaacctataccagcaccgcggtggacaccaacaaagatgttatcattgaggcgagcgtggaaccggcgaccgagcaagaaatcgcggacaccgtgaccgttatgggcggtcaggattgggaactgtggattcaagcgctggaggaagcgggcgttctggcggagggttgcaagaccgtggcgtacagctatatcggtaccgaactgacctggccgatttactgggacggcgcgctgggtcgtgcgaaaatggacctggatcgtgcggcgaccgcgctgaacgagaagctggcggcgaaaggcggtaccgcgaacgtggcggttctgaagagcgtggttacccaggcgagcagcgcgatcccggttatgccgctgtatattgcgatggtgttcaagaaaatgcgtgaacaaggcgttcacgagggttgcatggaacagatctaccgtatgtttagccaacgtctgtataaagaggacggcagcgcgccggaagtggacgatcacaaccgtctgcgtctggacgattgggagctgcgtgacgatatccagcaacactgccgtgatctgtggccgcaaattaccaccgagaacctgcgtgaactgaccgactacgatatgtataaagaggagttcatcaaactgttcggctttggtattgagggtattgactacgatgcggatgtgaacccggaagttgagtttgatgtgattgatattgagtaaaccggtctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatggctcgacagatct

SEQ ID NO: 4 - DNA, 17 bp, synthetic construct
  source 1..17: mol_type = other DNA
ccggccctgaaccagtt

SEQ ID NO: 5 - DNA, 21 bp, synthetic construct
  source 1..21: mol_type = other DNA
gtagccaagtgggagaaagct

SEQ ID NO: 6 - DNA, 16 bp, synthetic construct
  source 1..16: mol_type = other DNA
tcgaagtggtggccag

SEQ ID NO: 7 - DNA, 19 bp, synthetic construct
  source 1..19: mol_type = other DNA
cagcgctttcagcaattgc

SEQ ID NO: 8 - DNA, 20 bp, synthetic construct
  source 1..20: mol_type = other DNA
gaycggagcgatgcatagtc

SEQ ID NO: 9 - DNA, 15 bp, synthetic construct
  source 1..15: mol_type = other DNA
cccatatgacatccc

(19) United States
(12) Patent Application Publication
(10) Pub. No.: US 2026/0248901 A1
(43) Pub. Date: Aug. 27, 2026

(54) DNA VACCINE FOR FISH AGAINST SALMONID ALPHAVIRUS

(71) Applicant: INTERVET INC., Rahway, NJ (US)

(72) Inventors: Stephane VILLOING, Boxmeer (NL); Martijn Alexander LANGEREIS, Boxmeer (NL); Christiaan VOSSEN, Boxmeer (NL)

(73) Assignee: INTERVET INC., Rahway, NJ (US)

(21) Appl. No.: 19/160,357

(22) PCT Filed: Feb. 29, 2024
(86) PCT No.: PCT/EP2024/055266

§ 371 Date: Aug. 27, 2025

(30) Foreign Application Priority Data
Mar. 1, 2023 (EP) 23159357.5

(51) Int. Cl. A61K 39/12; A61K 39/00; A61P 31/14; C07K 14/005; C12N 7/00; C12N 9/02; C12N 15/85

(52) U.S. Cl. CPC A61K 39/12; A61P 31/14; C07K 14/005; C12N 7/00; C12N 9/001; C12N 15/85; A61K 2039/53; A61K 2039/545; A61K 2039/552; C12N 2770/36122; C12N 2770/36134; C12N 2770/36151; C12Y 103/01009

(57) ABSTRACT

Salmonid alphavirus (SAV) is an important pathogen affecting the aquaculture of Salmonid fish. Vaccines based on DNA plasmids expressing a SAV antigen have been described. However these are difficult to prepare at large scale and at the desired quality level. Also, they are commonly administered at relatively high amounts of plasmid per animal dose. This makes such DNA vaccines less affordable for this market. The invention discloses an improved plasmid that allows effective use as a DNA vaccine against SAV, but at a much reduced amount of DNA per dose. The plasmid contains a gene encoding a bacterial enzyme: Fab.

### FIELD OF THE INVENTION

The present invention relates to the field of veterinary vaccines, more specifically to a DNA vaccine for fish against salmonid alphavirus (SAV). In particular the invention relates to a plasmid expressing an antigen from SAV and comprising a fab gene, and to medical uses of said plasmid as a DNA vaccine for fish against SAV.

### BACKGROUND OF THE INVENTION

As one of the ways to provide the growing human world population with a consistent source of animal protein, the raising of fish in captivity has grown rapidly over the last decades.

Of the various species of fish bred in aquaculture, Atlantic salmon (Salmo salar) have the highest value per animal and is produced by the most industrialised type of fish farming. The largest producers of farmed Atlantic salmon are in Norway and in Chile.

Inevitably the keeping of large numbers of animals in close proximity makes them vulnerable to infections. Of the various bacterial- and viral diseases that affect farmed Salmonids, salmonid alphavirus (SAV), also called: salmon pancreas disease virus (SPDV), has a strong negative impact on animal welfare and economy of operation. SAV causes Pancreas disease (PD) in salmon and sleeping disease in trout. These diseases may result in reduced growth and mortality of between 10-60% of the animals.

SAV is an enveloped virus that has a linear single-stranded RNA genome of positive sense of about 12 kb in size, and belongs to the genus Alphavirus, in the family Togaviridae (Villoing et al., 2000, J. of Virol., vol. 74, p. 173-183). Currently 7 genetic subtypes are identified: SAV1-SAV6 (Fringuelli, 2008, J. of Fish Diseases, vol. 31, p. 811-823), and SAV7 (Tighe et al., 2020, Dis. Aquat. Organ., doi: 10.3354/dao03546).

All these are closely related biologically, and all belong to the same serotype. Of these SAV2 and SAV3 are currently the most prominent SAV viruses in Norway. The various subtypes provide good cross-neutralisation against each other, and use of one SAV subtype to vaccinate against another is common. For example, the vaccine Aquavac® PD (MSD Animal Health) contains inactivated virus of an Irish SAV 1 strain, but is marketed for protection against all SAV subtypes. Similarly Clynav® (described below) contains the genetic information of a SAV2 strain, but is marketed for protection against SAV3.

The alphavirus genome expresses two open reading frames (ORFs), each encoding a polyprotein: the upstream ORF encodes a polyprotein comprising the non-structural proteins. The downstream ORF encodes, via a 26S sub genomic mRNA, the structural polyprotein (SP); this is auto-cleaved post-translationally into five separate proteins: envelope proteins E1, E2 and E3, the capsid protein (C), and protein 6K. The E1 and E2 proteins together form the spikes of SAV, of which E2 is the immunodominant part.

To provide adequate health care measures, the vaccination of fish against a variety of pathogens is now a common practice in aqua culture, and a large variety of vaccines for fish is commercially available these days. They are based on inactivated- or subunit antigens, or on life attenuated pathogens; see: Bedaker & Kole, Fundamentals of Fish Vaccination, Chapter 9, in: Vaccine Design: Methods and Protocols, Volume 2—Vaccines for Veterinary Diseases, in: Methods in Molecular Biology, vol. 2411, https://doi.org/10.1007/978-1-0716-1888-2_9, ed. Sunil Thomas, Humana press, ISSN 1064-3745.

A recent development is the use in fish of vaccines that are based on nucleic acids, in particular: on plasmids that express the gene for a protein antigen from a fish pathogen. These so called: ‘DNA vaccines’ can be quite effective, although their mechanism of action is not yet fully understood. Most likely the plasmids are taken up by specialised cells after inoculation, and the heterologous gene in the plasmid gets expressed from its eucaryotic promoter. These cells then present the antigen to the target's immune system which triggers an immune response against the antigen, and subsequently against the corresponding pathogen. A review on DNA vaccines for fish is e.g. R. Dalmo, 2017, J. of Fish Dis., vol. 41, p. 1-9.

A plasmid to be used as a DNA vaccine contains different elements in order to be effective in the two different stages of its use: for the use as a vaccine, the plasmid itself only acts as a carrier for the antigen gene that is to be expressed after inoculation into the animal target. For the expression of that antigen gene the plasmid contains a eucaryotic promoter that is active in the cells of the target.

On the other hand, for the use in the manufacturing stage wherein large amounts of the plasmid are to be produced by amplification in bacteria, the plasmid contains a bacterial origin of replication (ORI), to control its replication in the bacteria. Often the plasmid also contains a gene that allows to put a positive selection pressure on the plasmid's maintenance in the bacteria. That selection gene needs to be driven by a procaryotic promoter in order to be expressed in the bacterium.

Normally, the parts for bacterial replication and expression do not have a role in the eucaryotic target, and vice versa: the antigen gene and eucaryotic promoter have no effect during bacterial amplification.

Currently two DNA vaccines for fish have been registered and are commercially available, both from Elanco GmbH: Apex-IHN®, against infectious hematopoietic necrosis virus (IHNV), which was registered in Canada in 2005; and: Clynav® against SAV3, which was registered in Europe in 2017. Both constructs are based on the pUK21 plasmid, whereby in Apex-IHN the plasmid comprises the G-protein gene of IHNV, and in Clynav, the plasmid comprises the full SP of a marine SAV2 isolate. Both plasmids employ the human cytomegalovirus immediate early 1 gene promoter to drive the expression of the antigen gene, and both use Kanamycin resistance for selection during bacterial amplification. Also, both are safe and very effective DNA vaccines, see: Long et al. (2017, Dis. Aquat. Organ., vol. 122, p. 213-221), and Røsaeg et al. (2021, J. of Fish Dis., vol. 44, p. 1911-1924), respectively.

However the main issues with the veterinary use of DNA vaccines today are in making them available at the desired quality and quantity, and at an acceptable cost price.

WO 2004/026338 and WO 2014/041189 describe the development of the APEX-IHN- and Clynav plasmid-based DNA vaccines, respectively. Clynav comprises the plasmid ‘pUK-SPDV-poly2#1’.

When producing a plasmid in bacterial cells, it is common practice to apply some sort of phenotypic selection pressure during culturing, to assure that the plasmids stay present in the bacteria during their successive rounds of amplification. Much used are selection systems whereby the plasmid expresses—from a procaryotic promoter—a gene that provides a selective advantage to a bacterial host cell, e.g. resistance to an antibiotic such as ampicillin or kanamycin. Kanamycin resistance results from the expression by the plasmid of an aminoglycoside phosphotransferase gene, also: KanR gene, and is the most used selection today. This because ampicillin-resistance genes are no longer allowed to be used in medicinal products in many countries. A less common alternative is to apply one of several non-antibiotic selection methods, for example by having the plasmid express a gene for an enoyl-acyl reductase (ENR).

An ENR, also: enoyl-acyl carrier protein reductase, catalyses the last reduction step of the bacterial type II fatty acid synthesis pathway. The genes encoding an ENR are indicated as ‘fab’ genes, encoding ‘Fab’ proteins, as they are ‘fatty acid biosynthetic’ genes and -proteins. A further letter then indicates their specific type, e.g. fabI or fabV, etc., For a review see: Hopf et al. (2022, Frontiers in Microbiology, vol. 13, article 891610).

First studied was FabI, which enzyme turned out to be the target of a biocidal compound commonly used in a variety of consumer products: Triclosan (5-chloro-2-[2,4-dichlorophenoxy] phenol; a.k.a. Irgasan). It was discovered that a mutation in- or overexpression of the fabI gene, made the bacterium resistant to Triclosan. This principle was later used as a non-antibiotic, positive selection system for plasmid maintenance in (E. coli) bacteria, see: Goh & Good (2008, BMC Biotechnol., vol. 8, doi: 10.1186/1472-6750-8-61), and: WO 2009/011651.

While the fabI gene was initially described for E. coli, it was later found to be endogenous in many different bacteria. Also several mutants, homologs and variants of fabI have been identified or were generated, and these have also been used in a triclosan based selection system, e.g.: a mutated fabI gene: mfabI (Jang & Magnuson, 2013, Plos One, vol. 8, doi: 10.1371/journal.pone.0057075.g007); homologs of fabI are e.g.: fabV from Vibrio or Pseudomonas bacteria (Ali & Chew, 2015, Plos One, DOI: 10.1371/journal.pone.0129547; WO 2015/165840); fabV2 from Aeromonas (Khan et al., 2018, Plos One, vol. 13, e0192277); fabL from Bacillus (Heath et al., 2000, J. Biol. Chem., vol. 275, p. 40128-40133); or fabK from Streptococcus (Heath & Rock, 2000, Nature, vol. 406, p. 145-146); and a variant of fabI is: fabI2 (Khan et al., 2019, Nature-Scientific Reports, vol 9, Art. nr. 15401); etcetera.

Plasmids can be produced in suitable bacteria, e.g. in laboratory strains of E. coli K12 type cells such as DH5alpha, DH10b, or Top10. As such the technique to produce and then isolate plasmids from bacteria, so-called ‘mini-prep’ isolation, is one of the most basic skills in molecular biology. However in the pharmaceutical industry there is relatively little experience with the production of plasmids at very large (kilogram) scale, to a pharmaceutical quality grade, and under procedures of good manufacturing practice. Inevitably such manufacturing is much more laborious and cumbersome than anything done at laboratory scale. This makes that plasmids for use in a DNA vaccine are expensive to produce in the required large quantities, and at the required quality level. This is a problem, especially in the case of veterinary use for livestock animals (including aquaculture), because such animal husbandry is typically a large volume—low margin enterprise. Consequently, cost of goods is a critical parameter, and this applies equally to the veterinary vaccines used in that sector.

This is exacerbated by the fact that the SAV DNA vaccines described so far, have had to be administered to their targets in relatively large amounts per dose, in order to be effective: Xu et al. (2012, Vaccine, vol. 30, p. 3918-3928), using plasmids comprising SAV E1 or E2 genes, administered two doses of 20 μg plasmid to each fish; Sobhkhez et al. (2018, PLOS ONE, doi.org/10.1371/-journal.pone.0204924) testing plasmids comprising a SAV SP, administered one dose of 15 μg plasmid per fish; a same dose was used by Chang when testing different SAV DNA vaccines (2021, Chapter 12, in: Vaccine Design: Methods and Protocols, supra); Collins et al. (2021, Vaccines, vol. 9, doi.org/10.3390/vaccines9020163), also using plasmids comprising a SAV SP, even administered two doses of 25 μg plasmid per fish.

The use of such amounts of 15-50 μg/fish would be too expensive to be commercially feasible in aquaculture vaccination. That explains why APEX-IHN is registered for use in salmon at 10 μg per dose, and the Clynav vaccine is registered at the somewhat reduced dose of between 5.1 and 9.4 μg plasmid per dose (CVMP assessment report for CLYNAV (EMEA/V/C/002390/0000)). However the recommended dose of Clynav vaccine in practice, as indicated on its product package leaflet is between 6 and 9.4 μg plasmid/fish (https://www.ema.europa.eu/en/documents/product-information/clynav-epar-product-information_en.pdf, see SmPC, Annex II, B: package leaflet). Therefore, these remain to be very costly vaccines.

It is therefore an object of the present invention to overcome a disadvantage in the prior art, and to accommodate to this need in the field by providing a SAV DNA vaccine that is effective at a lower amount of plasmid per dose.

### DESCRIPTION OF THE INVENTION

Surprisingly it was found that this object can be met, and consequently one or more disadvantages of the prior art can be overcome, by providing a plasmid that expresses a SAV antigen, and comprises a gene encoding an enoyl-acyl reductase.

Plasmids were constructed that expressed the SAV SP from a eucaryotic promoter and contained a fab gene. These were very effective as a DNA vaccine in salmon against a SAV challenge infection, even when administered at an amount of plasmid DNA per dose that was 5 μg or much less. The commercial SAV DNA vaccine Clynav when tested side-by-side in the same experiments, was much less effective when used at the same very low amount per dose. The main difference between the new plasmids and the Clynav plasmid is in the presence of a fab gene.

This development allows the manufacture and application of more economical SAV DNA vaccines, as they are effective at lower amounts of plasmid DNA per dose. Also, existing SAV DNA vaccine plasmids can now be made more effective by providing them with a fab gene.

This advantageous effect is unexpected and could not have been predicted from any disclosure in the prior art. This because there are only relatively few publications on DNA vaccines in fish, and even less on SAV DNA vaccines. Also, there is no publication on a plasmid comprising both a SAV antigen gene and a fab gene; let alone on the use of such a plasmid as an economical DNA vaccine against SAV in fish.

Therefore in one aspect the invention relates to a plasmid comprising a gene encoding an antigen from salmonid alphavirus (SAV) and a eucaryotic promoter operably linked to said gene, characterised in that said plasmid also comprises a fab gene.

A “plasmid” is well-known to be a polynucleotide molecule of double stranded DNA, that can replicate extra-chromosomally in bacteria. In biotechnology, plasmids have been adapted to be the work-horse of molecular biology that allow the cloning and the expression of a wide variety of inserted genes and—elements. A subclass of such synthetic plasmids are so-called expression plasmids, which allow the expression of a foreign gene in procaryotic- or eucaryotic host cells. Such plasmids are commonly between 1 and 10 kbp in size. When intended for the expression of the gene of interest in mammalian cells, they use a eucaryotic promoter. A wide variety of expression plasmids are commercially available, such as from the plasmid families: pUC, pBR, pGEM, pCI, pcDNA, pEMBL, etcetera.

The term “comprising” (as well as variations such as “comprises”, “comprise”, and “comprised”) as used herein, intends to refer to all elements, and in any possible combination conceivable for the invention, that are covered by—or included in the text section, paragraph, claim, etc., in which this term is used, even if such elements or combinations are not explicitly recited; and not to the exclusion of any of such element(s) or combinations.

Therefore, any such text section, paragraph, claim, etc., can therefore also relate to one or more embodiment(s) wherein the term “comprising” (or its variants) is replaced by terms such as “consist of”, “consisting of”, or “consist essentially of”.

The term “gene” is used to indicate a section of nucleic acid that is capable of encoding a protein. For the invention a gene corresponds to an ‘open reading frame’ (ORF), i.e. a protein-encoding section of DNA, from start-to stop codon, not including the gene's promoter. A gene for the invention may encode a complete protein, or may encode a section of a protein, for example encoding only the mature form of a protein, i.e. without a ‘leader’, ‘anchor’, or ‘signal sequence’. A gene may even encode a specific section of a protein, e.g. a section comprising an immunoprotective epitope.

In this regard a “protein” for the invention is a molecular chain of amino acids. The protein can be a native or a mature protein, a pre- or pro-protein, or a functional fragment of a protein. Therefore peptides, oligopeptides and polypeptides are included within the definition of protein, as long as these still contain a relevant immunogenic epitope.

For the invention, the term “encoding” refers to the well-known principle of gene expression wherein genetic information provides the code for the production of a protein, via transcription and translation.

A “salmonid alphavirus” (SAV) refers to a species of virus in the taxonomic genus Alphavirus. Such a virus has the characterising features of its taxonomic group, such as the morphologic-, genomic-, and biochemical characteristics, as well as the biological characteristics such as the physiologic-, immunologic-, and pathologic behaviour. SAV infects especially fish of the Salmonidae family (see details below). The characteristics and the effects of SAV in aquaculture are reviewed e.g. in McLoughlin & Graham (2007, J. Fish Dis., vol 30, p. 511-531).

As is known in the field, the classification of a micro-organism in a particular taxonomic group is based on its combined features. The invention therefore also includes variants in the SAV species that are sub-classified therefrom in any way, for instance as a subspecies, strain, isolate, genotype, variant, subtype, or subgroup, and the like. For example, several genetic subtypes of SAV have been described and are currently named SAV1 through SAV7.

Further, it will be apparent to a person skilled in the art in the field of the invention that while a particular SAV for the invention may currently be assigned to a species or subtype, however that is a taxonomic classification that could change in time as new insights can lead to reclassification into a new or different taxonomic group. However, as this does not change the virus itself, or its antigenic repertoire, but only it's scientific name or classification, such re-classified viruses remain within the scope of the invention.

An “antigen” for the invention is a protein that is recognised by the immune system of the vaccinated target and induces an immunogenic response from the humoral- and/or the cellular immune system of the target, against that antigen. As the antigen is derived from a fish pathogen, an immune response against the antigen also affects that pathogen.

For the invention, the terms “eucaryotic”- or “prokaryotic” promoter refer to promoters that are active in eucaryotic- or in procaryotic host cells, respectively. Clearly that does not mean that the promoter element itself needs to be derived from such an organism, for example it can also be derived from a micro-organism active in such an organism such as a bacteriophage or a eucaryotic virus, or can be of synthetic origin.

A “promoter” is well-known to be a functional genetic element that directs the transcription of a downstream coding region. A promoter is thus situated upstream of a gene.

The nomenclature of a promoter is commonly based on the gene of which it controls the expression in its natural context. For example, the “hCMV-IE1 gene promoter” as used herein, refers to the promoter that in nature drives the expression of the IE1 gene from human CMV, and is thus situated immediately upstream of the IE1 gene in the hCMV genome.

Commonly promoters contain a number of recognisable, regulatory regions, such as the enhancer region, which is involved in binding regulatory factors that influence the timing, the duration, the conditions, and the level of transcription. While the enhancer region is commonly situated upstream of a promoter, a promoter can also be influenced by regions more downstream towards the start codon, that are involved in the binding of transcription factors and in directing the RNA polymerase itself. Examples are conserved sequence elements such as: the TATA box, the CAAT box, and the GC box.

A promoter typically is “operably linked” to the gene of which it drives the expression. That means the promoter and the gene are in close proximity, and no significant other sequences are present between them that would intervene with an effective start of the transcription.

A “fab” gene for the invention is a gene that encodes an ENR. As described, an ENR is a bacterial enzyme involved in fatty acid biosynthesis. The ENR enzyme has EC number 1.3.1.9.

The initial fab gene described was fabI. Since then several mutants, homologs and variants of the fabI gene were described, e.g.: mfabI, fabV and fabI2.

The generation, construction, and assembly of the plasmid according to the invention can be done by well-known molecular biological techniques, involving cloning, transfection, recombination, selection, and amplification. These, and other techniques, are explained in great detail in standard text-books like Sambrook & Russell: “Molecular cloning: a laboratory manual” (2001, Cold Spring Harbour Laboratory Press; ISBN: 0879695773); Ausubel et al., in: Current Protocols in Molecular Biology (J. Wiley and Sons Inc, NY, 2003, ISBN: 047150338X); C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421).

Details of embodiments and of further aspects of the invention will be described below.

### SAV:

In an embodiment of the plasmid according to the invention the SAV for the invention is selected from SAV1, SAV2, SAV3, SAV4, SAV5, SAV6 and SAV7. More preferably the SAV is selected from SAV1, SAV2, and SAV3; even more preferably the SAV is selected from SAV2 and SAV3. Most preferably the SAV is SAV3.

Therefore in an embodiment of the plasmid according to the invention, the SAV is selected from SAV1, SAV2, and SAV3.

Samples of SAV for use in the invention can be obtained from a variety of sources, e.g. as field isolate from a fish in the wild or from aquaculture, or from various laboratories, (depository) institutions, or (veterinary) universities. Also, much genetic information on SAV is available digitally in public sequence databases such as NCBI's GenBank™, and EMBL's EBI™.

The genetic subtypes of SAV have been defined by Fringuelli (supra). SAV1 is further described in Nelson et al. (1995, Dis. Aquat. Organ., vol, 22, p. 25-32). SAV2 is further described in Castric et al. (1997, Bull. of the Eur. Ass. of Fish Path., vol. 17, p. 27-30). SAV3 is further described by Hodneland et al. (2005, Dis. Aquat. Organ., vol. 5, p. 113-20; with Erratum in: 2005, Dis. Aquat. Organ., vol. 67, p. 181).

Antigen from SAV:

In an embodiment of the plasmid according to the invention, the antigen from SAV is the structural polyprotein (SP) of SAV.

The SP includes all of the proteins: C, E3, E2, 6k and E1. The SP polyprotein upon expression in the vaccinated target will auto-cleave into the separate SAV structural proteins, which then form different subunits and complexes. When these are recognised by the target's immune system, that will generate an immune response against SAV.

The SAV SP is about 1320 amino acids long, with some size variation between isolates, mainly in the C- and the 6K proteins.

In a preferred embodiment the SAV SP for the invention is the SP from a SAV selected from SAV1, SAV2, and SAV 3; more preferably the SP is selected from SAV2 and SAV3; most preferred the SP is from SAV3.

For the invention, genes encoding a SAV SP for the invention are well known and are described in publications. Also their sequence information is readily available in public databases.

In a preferred embodiment, the gene encoding the SAV SP is the region from nucleotide 1078 up to and including nucleotide 5034 from SEQ ID NO: 3.

In a yet more preferred embodiment the gene encoding the SAV SP is operably linked at its 3′ end to a terminator and a polyA signal. Even more preferably, the terminator and polyA signal are provided as an SV40 late terminator-poly A signal; still even more preferably, the terminator and polyA signal is the region from nucleotide 5061 up to and including nucleotide 5182 from SEQ ID NO: 3.

### Eucaryotic Promoter:

In an embodiment of the plasmid according to the invention the eucaryotic promoter is a human CMV (hCMV) IE1 gene promoter.

The IE1 gene promoter from CMV is also called the major IE promoter of CMV. Details and sequences of this promoter are well known and readily available, e.g. from a wide variety of commercial eucaryotic expression plasmids, and digitally from public sequence databases.

Preferably the IE1 gene promoter is from human CMV.

In a preferred embodiment the hCMV IE1 gene promoter comprises an enhancer sequence.

More preferably, the eucaryotic promoter for the invention comprising the hCMV IE1 gene promoter and enhancer, is the region from nucleotide 48 up to and including nucleotide 742 from SEQ ID NO: 3.

### FAB Gene:

In an embodiment of the plasmid according to the invention the fab gene is fabI or is a mutant, homolog, or variant of fabI.

As is well-known, the fabI gene for the invention can be derived from any of a large number of bacteria. Preferably the fabI gene is derived from E. coli.

Many such sequences are publicly available, e.g. the fabI gene sequence from the E. coli K12 genome is available in GenBank acc. nr. CP064682, where the fabI gene is the complement of nucleotides 1344508-1345296. This section, already in a complemented and reversed format, is presented in SEQ ID NO: 1.

A “mutant of fabI” for the invention is a gene encoding an ENR but which gene has been naturally or artificially changed so that it is no longer the same as a fabI gene for the invention. An example is the mfabI gene.

In an embodiment a mutant fab gene has a nucleotide sequence that has at least 97% nucleotide sequence identity to the full length of SEQ ID NO: 1. More preferred is a nucleotide sequence identity of at least 98, or even 99%, in that order of preference, to the full length of SEQ ID NO: 1.

For determining the percentage of nucleotide sequence identity for the invention the option ‘blastn’ is selected from the Blast™ computer program, i.e. the NCBI's Basic Local Alignment Search Tool, and the “Align two or more sequences” option is selected, with standard settings and default parameters (http://blast.ncbi.nlm.nih.gov/Blast.cgi).

A “homolog of fabI” for the invention is a gene encoding an ENR but which is taxonomically classified with a different name indicator. Examples are the fabK, fabL, and fabV genes.

A ‘variant’ of a fabI or of a homolog of fabI, is e.g. a fabI2- or a fabV2 gene.

Examples of such fab genes are well known from the prior art, and their sequences are described in public databases. For example, but not limited to: further fabI genes are well known from bacterial genera such as Bacillus, Klebsiella, Pseudomonas, Shigella; fabK is e.g. well known from bacterial genera such as Serratia, Streptococcus, Clostridium, Enterococcus; fabL is e.g. well known from bacterial genera such as Chlamydia, Fusobacterium, Listeria, Legionella; and fabV is e.g. well known from bacterial genera such as Vibrio, Xanthomonas, Streptomyces, Erysipelothrix.

An example of a fabV2 gene for the invention is in GenBank acc. nr.: BBE13260.

In a preferred embodiment of the plasmid according to the invention the fab gene is selected from: fabI, fabK, fabL, fabV, mfabI, fabI2, fabL2, and fabV2.

In an embodiment a homolog of a fabI gene has a nucleotide sequence that has at least 90% nucleotide sequence identity to the full length of SEQ ID NO: 1. More preferred is a nucleotide sequence identity of at least 92, 94, 95, 96, 97, 98, or even 99%, in that order of preference, to the full length of either SEQ ID NO: 1.

To improve potential fab gene expression in bacteria during the amplification of the plasmid, the fab gene can be codon optimised.

As is well known in the art it can be beneficial for the expression of a gene to adapt its codon-usage. This involves the adaptation of the nucleotide sequence of the gene to be expressed, to encode the intended amino acids, but by way of a nucleotide sequence that better matches the codon preference (the tRNA repertoire) of the target cells in which it is to be expressed. Consequently, the nucleotide mutations applied are commonly ‘silent’. Such modifications are commonly planned in silico by using one of many available computer software programs, after which the desired nucleotide sequence can be synthesized.

Therefore, in a more preferred embodiment, the fab gene for the invention is codon optimised towards the codon use table of E. coli.

In an even more preferred embodiment, the fab gene for the invention is selected from
the fabV2 gene that is the region from nucleotide 6329 up to and including nucleotide 7534 from SEQ ID NO: 3, and
from the fabI gene that is the region from nucleotide 6294 up to and including nucleotide 7082 from SEQ ID NO: 2.

Most preferred is the fabI gene that is the region from nucleotide 6294 up to and including nucleotide 7082 from SEQ ID NO: 2;

Preferably, the plasmid according to the invention not only comprises the fab gene for the invention, but also expresses that fab gene in a bacterial host cell, by way of a prokaryotic promoter that is operatively linked to said fab gene. This will allow to apply non-antibiotic phenotypic selection, to maintain the plasmid according to the invention in the bacteria during plasmid amplification.

Therefore in an embodiment, the plasmid according to the invention expresses the fab gene from a prokaryotic promoter that is operatively linked to said fab gene.

The prokaryotic promoter to drive the fab gene for the invention can be any suitable prokaryotic promoter.

In a preferred embodiment of the plasmid according to the invention wherein the fab gene is expressed in bacteria from a prokaryotic promoter, said promoter is from a chloramphenicol O-acetyltransferase gene; also: a CatR gene. The CatR gene as such would provide a bacterium with resistance against chloramphenicol, however that function is not transferred to the plasmid according to the invention, when only employing the CatR gene promoter.

CatR genes and their promoters are well known in the art and are readily available, for example from the region that is the reverse-complement of the nucleotides 5882-7065, from GenBank acc.nr. KX273378.

More preferably the CatR gene promoter is the region from nucleotide 6191 up to and including nucleotide 6293 from SEQ ID NO: 2.

In an alternate preferred embodiment of the plasmid according to the invention wherein the fab gene is expressed, the prokaryotic promoter to drive the fab gene is a promoter of a KanR gene. KanR genes and their promoters are well known in the art and are readily available.

More preferably the KanR gene promoter is the region from nucleotide 6191 up to and including nucleotide 6328 from SEQ ID NO: 3.

### Plasmid:

In an embodiment, the plasmid according to the invention is based on a plasmid selected from the group of pUK, pBR, pGEM, and pCI plasmids, or on a variant of one of those plasmids.

Preferably the plasmid according to the invention is based on a pUK or a pCI plasmid; more preferably is based on a pCI plasmid.

Therefore in an embodiment of the plasmid according to the invention, the plasmid is based on a pCI plasmid.

Such plasmids are commercially available from a large number of suppliers, and their sequences are public, e.g.: pUK21 is in GenBank acc. nr. AF223640, and pCI is in GenBank acc.nr. U47119. The pCI plasmid is available commercially e.g. from Promega Corp. Both plasmids pUK21 and pCI comprise the eucaryotic hCMV IE1 gene promoter.

In a preferred embodiment of the plasmid according to the invention, the pCI plasmid is as described in GenBank acc.nr. U47119.

For the invention, “based on a plasmid” means that the plasmid according to the invention was constructed starting with the indicated parental plasmid, which was subsequently modified by some mutation, insertion, and/or deletion. At least maintained from the parental plasmid are the ORI, and preferably also the eucaryotic promoter it comprises.

In an embodiment of the plasmid according to the invention, the plasmid also comprises an aminoglycoside phosphotransferase (KanR) gene, and a procaryotic promoter that is operably linked to said KanR gene. This will allow phenotypic selection of plasmid maintenance by Kanamycin resistance.

Preferably the KanR gene is derived from Tn903 (Oka et al., J. of Mol. Biol., vol. 147, p. 217-226); more preferably the KanR gene has the sequence of GenBank acc. nr. V00621.

Preferably the procaryotic promoter driving the KanR gene is a native KanR gene promoter.

In case the plasmid according to the invention expresses a gene allowing phenotypic selection of plasmid maintenance, e.g. a KanR gene, the plasmid would not need to, although it still could, express the fab gene for the invention. So while the plasmid according to the invention needs to contain a fab gene to be effective for the invention, in the situation described in the previous sentence, that plasmid would not need to express that fab gene, and therefore would not need to comprise a promoter that is operatively linked to that fab gene.

Alternatively, in case the plasmid according to the invention expresses the fab gene for the invention, the plasmid would not need to, although it still could, express a further gene allowing phenotypic selection of plasmid maintenance, e.g. express a KanR gene.

In a preferred embodiment of the plasmid according to the invention, the plasmid expresses the fab gene, and expresses no other gene for phenotypic selection. More preferably, the plasmid expresses the fab gene, and no other gene for phenotypic selection is even comprised in the plasmid according to the invention.

In an embodiment of the plasmid according to the invention, one or more of the conditions apply, selected from the group consisting of:
the SAV for the invention is selected from SAV1, SAV2, SAV3, SAV4, SAV5, SAV6 and SAV7;
the SAV is selected from SAV1, SAV2, and SAV3;
the SAV is selected from SAV2 and SAV3;
the SAV is SAV3;
the antigen from SAV is the structural polyprotein (SP) of SAV;
the SAV SP is the SP from a SAV selected from SAV1, SAV2, and SAV 2;
the SAV SP is the SP from a SAV selected from SAV2 and SAV3;
the SAV SP is the SP from SAV3;
the gene encoding the SAV SP is the region from nucleotide 1078 up to and including nucleotide 5034 from SEQ ID NO: 3;
the gene encoding the SAV SP is operably linked at its 3′ end to a terminator and a polyA signal;
the terminator and polyA signal is a SV40 late terminator-poly A signal; preferably, the terminator and polyA signal is the region from nucleotide 5061 up to and including nucleotide 5182 from SEQ ID NO: 3;
the eucaryotic promoter is an hCMV IE1 gene promoter;
the hCMV IE1 gene promoter comprises an enhancer sequence;
the eucaryotic promoter is the region from nucleotide 48 up to and including nucleotide 742 from SEQ ID NO: 3;
the fab gene is fabI or is a mutant, homolog, or variant of fabI;
the fabI gene is derived from E. coli;
the mutant fab gene has a nucleotide sequence that has at least 97% nucleotide sequence identity to the full length of SEQ ID NO: 1;
the mutant fab gene has a nucleotide sequence identity of at least 98, or even 99%, in that order of preference, to the full length of either SEQ ID NO: 1;
the homolog of a fabI gene has a nucleotide sequence that has at least 90% nucleotide sequence identity to the full length of SEQ ID NO: 1;
the homolog of a fabI gene has a nucleotide sequence identity of at least 92, 94, 95, 96, 97, 98, or even 99%, in that order of preference, to the full length of either SEQ ID NO: 1;
the fab gene is selected from: fabI, fabK, fabL, fabV, mfabI, fabI2, fabL2, and fabV2;
the fab gene is codon optimised towards the codon use table of E. coli;
the fab gene is selected from the fabV2 gene that is the region from nucleotide 6329 up to and including nucleotide 7534 from SEQ ID NO: 3; and from the fabI gene that is the region from nucleotide 6294 up to and including nucleotide 7082 from SEQ ID NO: 2;
the fab gene is the fabI gene that is the region from nucleotide 6294 up to and including nucleotide 7082 from SEQ ID NO: 2;
the plasmid expresses the fab gene from a prokaryotic promoter that is operatively linked to said fab gene;
the prokaryotic promoter to drive the fab gene is a promoter of a chloramphenicol O-acetyltransferase (CatR) gene;
the CatR gene promoter is the region from nucleotide 6191 up to and including nucleotide 6293 from SEQ ID NO: 2;
the prokaryotic promoter to drive the fab gene is a promoter of an aminoglycoside phosphotransferase (KanR) gene;
the KanR gene promoter is the region from nucleotide 6191 up to and including nucleotide 6328 from SEQ ID NO: 3;
the plasmid is based on a plasmid selected from the group of pUK, pBR, pGEM, and pCI plasmids, or on a variant of one of those plasmids;
the plasmid is based on a pUK or a pCI plasmid;
the plasmid is based on a pCI plasmid;
the plasmid also comprises a KanR gene and a procaryotic promoter that is operably linked to said KanR gene;
said KanR gene is derived from Tn903 (Oka et al., J. of Mol. Biol., vol. 147, p. 217-226); preferably the KanR gene has the sequence of GenBank acc. nr. V00621;
the procaryotic promoter driving the KanR gene is a native promoter of an aminoglycoside phosphotransferase (KanR) gene;
the plasmid expresses the fab gene, and expresses no other gene for phenotypic selection; and
the plasmid expresses the fab gene, and no other gene for phenotypic selection is comprised in the plasmid.

In Tables 1 and 2 below, the main features of two embodiments of a plasmid according to the invention are described: ‘pCI-SAV3 SP-Cat prom-fabI’ (SEQ ID NO: 2) and ‘pCI-SAV3 SP-Kan prom-fabV2’ (SEQ ID NO: 3).

TABLE 1
The main features of SEQ ID NO: 2 (pCl-SAV3 SP-Cat prom-fabl)

| | |
|---|---|
| Nucleotide region | Main elements of SEQ ID nr. 2 |

| | | |
|---|---|---|
| 48 | 742 | hCMV IE1 gene promoter |
| 1078 | 5037 | SAV3 SP |
| 5061 | 5182 | SV40 terminator-polyA signal |
| 6191 | 6293 | CatR gene promoter |
| 6294 | 7082 | fabl gene |

TABLE 2
The main features of SEQ ID NO:
3 (pCl-SAV3 SP-Kan prom-fabV2)

| | |
|---|---|
| Nucleotide region | Main elements of SEQ ID nr. 3 |

| | | |
|---|---|---|
| 48 | 742 | hCMV IE1 gene promoter |
| 1078 | 5034 | SAV3 SP |
| 5061 | 5182 | SV40 terminator-polyA signal |
| 6191 | 6328 | KanR gene promoter |
| 6329 | 7534 | fabV2 gene |

In a preferred embodiment of the plasmid according to the invention, the plasmid has a nucleotide sequence that has at least 90% nucleotide sequence identity to the full length of SEQ ID NO: 2, or of SEQ ID NO: 3.

Still more preferred is a nucleotide sequence identity of at least 91, 92, 93, 94, 95, 96, 97, 98, or even 99%, in that order of preference, to the full length of either SEQ ID NO: 2, or of SEQ ID NO: 3.

In a more preferred embodiment the plasmid according to the invention has the nucleotide sequence of SEQ ID NO: 2.

In an alternate more preferred embodiment the plasmid according to the invention has the nucleotide sequence of SEQ ID NO: 3.

As is apparent from SEQ ID NOs: 2 and 3, there are further sequence elements comprised in the plasmids according to the invention besides the ones listed in Tables 1 and 2. Such further sequences may be remnants of the construction process, or may serve to assist in the replication or in the construction of said plasmid, such as: the Origin of replication, sites for restriction enzyme recognition, or PCR cloning sites.

As will also be apparent to a skilled person, the inserted genes in the plasmids according to the invention and their operatively linked promoters together, form self-contained expression units, therefore their orientation in the plasmid is not critical. SEQ ID NOs: 2 and 3 in that respect each only present one of the two possible orientations in those plasmids of the SAV SP-, the CatR-, and the KanR gene.

The plasmid according to the invention can be amplified in a procaryotic host cell, typically a bacterium, in a culture in vitro.

Therefore in a further aspect the invention regards a host cell comprising the plasmid according to the invention.

In an embodiment, the host cell according to the invention is a prokaryotic cell; preferably the host cell is a bacterium; more preferably the bacterium is E. coli.

The plasmid according to the invention can conveniently be amplified in-, and harvested from the host cell according to the invention, using methods and materials well known in the art.

As described, the unexpected advantageous effect of the plasmid according to the invention is prominent when the plasmid is applied as a DNA vaccine against SAV.

Therefore in a further aspect the invention relates to the plasmid according to the invention for use as a DNA vaccine for fish against infection and/or disease caused by SAV.

Details and examples of such a use as a DNA vaccine are described and exemplified herein.

Also, in a further aspect the invention relates to a DNA vaccine, said vaccine comprising the plasmid according to the invention and a pharmaceutically acceptable carrier.

And in still a further aspect, the invention relates to a DNA vaccine according to the invention for use in fish against infection and/or disease caused by SAV.

A “vaccine” is well-known to be a composition that can induce the immune system of a target inoculated therewith to launch a protective immunological response by the humoral- and/or by the cellular route, against a pathogen. Consequently, a vaccine for the invention is an immunogenic composition.

In the present case the vaccine is a DNA vaccine, and the plasmid it comprises can expresses the SAV antigen for the invention, after inoculation into the fish target. The DNA vaccine according to the invention then induces protection in the inoculated fish against infection and/or disease caused by SAV. This protection is obtained by preventing or reducing the establishment or the proliferation of a productive infection by SAV virus in target organs. This is achieved for example by reducing the SAV viral load or shortening the duration of the viral replication. In turn this leads to a reduction in the target fish of the number, the intensity, and/or the severity of clinical signs of disease caused by the SAV infection.

For the invention, vaccination-efficacy, i.e. the protection induced by the vaccine according to the invention, is expressed by measuring the reduction of challenge virus viremia. This is expressed as relative percent protection (RPP) of the test group compared to the negative control group, as is outlined in detail below.

RPP is a good practical indication for vaccination efficacy, as it embraces all types of immune protection against SAV infection and/or disease, by the vaccine according to the invention: via the cellular route, by cytotoxic T cells; and via the humoral route, by inducing specific antibodies, either virus-neutralising antibodies, or antibodies that are non-neutralising but inducing complement-mediated cytolysis. RPP is defined as:

{1−(% PCR+fish in vaccinated group/% PCR+fish in unvaccinated group)}×100.

For the invention the percentage PCR positive animals is determined by RT PCR on fish sera at 3 weeks post challenge. Details are provided in the Examples.

In embodiments of the DNA vaccine according to the invention, and of the DNA vaccine for use according to the invention, the vaccine (for use) achieves an RPP of at least 55%.

Preferably, the DNA vaccine, and the DNA vaccine for use, both according to the invention, achieve an RPP of at least 60, 65, 70, 75, 80, 85, 90, or even 95% RPP, in this order of preference.

Methods to prepare and formulate a DNA vaccine for the invention are well-known, for example from handbooks such as: “Remington: the science and practice of pharmacy” (2000, Lippincott, USA, ISBN: 683306472), and: “Veterinary vaccinology” (P. Pastoret et al. ed., 1997, Elsevier, Amsterdam, ISBN 0444819681).

The application of a DNA vaccine for use according to the invention, in the vaccination of fish against infection and/or disease caused by SAV, is well within the skills of the routine practitioner. The efficacy of such application is prominent from the immunological response following vaccination, e.g. from the reduction of clinical symptoms or mortality after a challenge infection, and from scoring the vaccinated fish's signs of disease, clinical scores, serological parameters, or by re-isolation of the challenge pathogen; and comparing these results to a challenge-response in unvaccinated animals. For SAV the main parameters of infection and disease in fish are characteristic histological changes, including severe degeneration, necrosis, and inflammation in the pancreas, -heart, and -skeletal muscle. SAV vaccine efficacy can be determined e.g. by establishing in the vaccinated target a reduction of challenge virus viraemia, and a reduction of specific lesions in heart, muscle and/or pancreas.

The protection against SAV-induced infection and/or disease by the plasmid for use, and by the DNA vaccine for use, both according to the invention, results in the vaccinated fish targets in an improvement of health and of economic performance. This can for instance be assessed from parameters such as increase of well-being, of survival, and of growth rate, reduction of costs for veterinary healthcare, and increase of the economy of operation.

A “pharmaceutically acceptable carrier” is intended to aid in the stabilisation and administration of the vaccine, while being harmless and well-tolerated by the target. Such a carrier can for instance be sterile water or a sterile physiological salt solution. In a more complex form the carrier can e.g. be a buffer, which can comprise further additives, such as stabilisers or preservatives.

A preferred pharmaceutically acceptable carrier for the vaccine according to the invention is a buffer such as phosphate-buffered saline (PBS), or a ‘virus dilution buffer’ having 150 mM NaCl, 15 mM MgCl2, and 10 mM Tris, at pH 8.2.

In an embodiment, the DNA vaccine according to the invention may comprise a compound to provide for the delivery and/or the stabilisation of the plasmid according to the invention. Well-known compounds for this purpose are lipid particles such as liposomes and lipid nano particles; cationic polymers; dendrimers; alginate particles; and poly-lactic acid particles.

In the DNA vaccine according to the invention, the plasmid according to the invention is comprised in an amount that is immunologically effective. Because of the present invention, the effective amount of the plasmid according to the invention is lower than the amount of the plasmid used in prior art SAV DNA vaccines.

Therefore in an embodiment, the DNA vaccine according to the invention comprises the plasmid according to the invention in an amount that is 5 micrograms per animal dose or less. Preferably the DNA Vaccine comprises the plasmid in an amount that is 4, 3, 2, 1, 0.8, 0.6, 0.4, or even 0.3 micrograms per animal dose or less, in this order of preference.

For the invention, the total DNA concentration of a sample of the plasmid according to the invention is determined by way of UV photometry at 260 nm. Suitable apparatus is e.g. the NanoDrop™ from ThermoFisher.

The DNA vaccine according to the invention can be administered to a fish by different routes. In an embodiment, the DNA vaccine according to the invention is administered by parenteral route, i.e. through the skin, e.g.: intramuscular, intraperitoneal, intradermal, submucosal, or subcutaneous. Preferred route of administration is by intradermal-, intramuscular- or subcutaneous route; Most preferred is intramuscular administration.

The volume per dose of the vaccine according to the invention can be selected according to the characteristics of the specific vaccine applied, the characteristics of the target, and the intended route of application. Parenteral injection of fish is commonly done with a dose of 0.01-1 ml/target. Preferably the dose is between 0.01 and 0.1 ml/target. More preferably the dose is selected from 10, 25 and 50 microliters/target.

The DNA vaccine according to the invention can be used both as a prophylactic- and as a therapeutic treatment, as it interferes with the establishment- and with the progression of an infection by SAV in a fish.

The DNA vaccine according to the invention can serve as an effective priming vaccination, which can later be followed and amplified by a booster vaccination, with the same or with a different vaccine.

The method, timing, dose and volume of the administration of the DNA vaccine according to the invention can be adapted and optimised for the particular type of fish to be vaccinated; also taking into consideration the time and life stage when the fish could be exposed to the SAV pathogen.

For Atlantic salmon, the administration is preferably performed in the so-called ‘smolt’ stage, shortly before the transfer to salt water.

The dosing regimen for administering the DNA vaccine according to the invention to a target organism can be in single- or in multiple doses, in a manner compatible with the formulation of the vaccine, and with the animal husbandry particulars of the target fish, and in such an amount as will be immunologically effective.

Preferably the DNA vaccine according to the invention is given only once, i.e. is a single shot vaccine.

Ideally, the regimen for the administration of the DNA vaccine according to the invention is integrated into existing vaccination schedules of other vaccines that the target fish may require, in order to reduce stress to the animals, and to reduce labour costs. These other vaccines can be administered in a simultaneous-, concurrent-, or sequential fashion, or by so-called: ‘associated use’; preferably these combinations are applied in a manner compatible with the licensed use of these vaccines.

For example, it is already common practice to combine the administration of a DNA vaccine to salmon smolts, with the administration of an oil-adjuvated emulsion vaccine. For that purpose automated vaccination equipment is available that vaccinates individual salmon with two needles: one administering a DNA vaccine intramuscularly in the side of the fish, and one administering an emulsion vaccine intraperitoneally in the fish's abdomen.

Therefore in a further aspect the invention regards a method for the protection of fish against infection and/or disease caused by SAV, the method comprising the step of administering to said fish the DNA vaccine according to the invention, or the DNA vaccine for use according to the invention.

A “fish” for the invention is an aquatic organism, with fins and gills, and can be a cartilaginous- or bony fish.

In embodiments of the plasmid for use according to the invention, of the DNA vaccine for use according to the invention, and of the method for the protection of fish according to the invention, the fish is a Salmonid.

A “Salmonid” fish is a bony fin fish of the family Salmonidae. This family comprises fish such as salmon, trout, char, freshwater whitefish, and graylings.

In a preferred embodiment the Salmonid for the invention is selected from salmon, trout, and char.

Even more preferably the char is Arctic char (Salvelinus alpinus); the salmon is selected from: Atlantic-, steelhead-, chinook-, coho-, pink-, chum-, and sockeye salmon; and the trout is selected from: rainbow-, Adriatic-, flathead-, marble-, ohrid-, sevan-, brook-, lake-, and brown trout.

Yet even more preferably the Salmonid is selected from: a fish from the genus Salmo, and a rainbow trout.

For the invention, these names of fish are to be interpreted in the same way as indicated above, namely that they are taxonomic classifications that could change in time as new insights can lead to reclassification into a new- or different taxonomic group. However, as this does not change the fish itself but only it's scientific name or classification, such re-classified fish remain within the scope of the invention. This includes any subtypes, variants, cross-breeds or hybrids of these fish for the invention.

Many possibilities exist for the manufacture of the DNA vaccine according to the invention. General techniques and considerations that apply to the manufacture of vaccines under well-known standards for pharmaceutical production are described for instance in governmental directives and regulations (Pharmacopoeia, 9CFR) and in well-known handbooks (“Veterinary vaccinology” and: “Remington”, both supra). Commonly such vaccines are prepared sterile, and are prepared using excipients of pharmaceutical quality grade.

Such preparations will incorporate microbiological tests for sterility, and absence of extraneous agents; they may also include studies in vivo or in vitro for confirming efficacy and safety. After completion of the testing for quality, quantity, sterility, safety and efficacy, the vaccine can be released for sale. All these are well known to a skilled person.

Therefore in a further aspect the invention regards the use of the plasmid according to the invention for the manufacture of the DNA vaccine according to the invention, or of the DNA vaccine for use according to the invention.

Also, in a further aspect the invention regards a method for the preparation of the DNA vaccine according to the invention, or of the DNA vaccine for use according to the invention, said method comprising the step of admixing a plasmid according to the invention and a pharmaceutically acceptable carrier.

The advantageous effect of the invention is thus: the increase of dose-efficacy for a DNA vaccine against SAV, by comprising in the vaccine plasmid a fab gene. It is now feasible to apply this improvement also to other DNA vaccines against SAV. This can conveniently be achieved by inserting in a vaccine's plasmid, a fab gene as described for the invention.

Therefore in a further aspect the invention regards a method to increase the efficacy of the plasmid in a SAV DNA vaccine, the method comprising the step of inserting a fab gene into said plasmid.

The ‘inserting’ for the invention regards well-known molecular cloning techniques. The inserting into the plasmid according to the invention can add to—or replace (a part of) the content of said plasmid.

Consequently the net to result of such inserting may be an addition, a substitution, or a deletion, as the skilled person will appreciate.

The invention is described herein in various aspects and embodiments. It should be understood that any combination of these are considered to be within the scope of the invention. However merely for conciseness, not every possible combination is outlined herein in full.

The invention will now be further described by the following, non-limiting, examples.

### EXAMPLES

### Example 1: Cloning of the SAV3 SP ORF

The DNA sequence encoding the SP polyprotein (capsid-E3-E2-6K-E1) was prepared from a Norwegian isolate of SAV3 (MSD AH: PD03.13, Haverøy) using classical techniques of total RNA isolation from low passage virus in supernatant of CHSE-214 cells; cDNA preparation; and high fidelity PCR amplification. This yielded 4 fragments together covering the complete SAV3 SP ORF. The primers used were based on the SAV3 strain N3 nucleotide sequence from GenBank acc. nr.: AY604237, and contained overlapping restriction enzyme sites (SaclI, NsiI, and EcoRV, originally present in the SAV cDNA sequence) to allow sequential ligations of the fragments. Also restriction sites EcoRI or NotI were included in the end primers, to allow subcloning of the complete fragment into the pCI plasmid as described below. The complete SP ORF insert was sequenced, and verified by comparison with sequences of SAV3 SP sequences available in GenBank. The resulting SAV3 SP ORF is as is indicated in Tables 1 and 2 above, and in SEQ ID NOs: 2 and 3.

This SAV3 SP ORF has 97% amino acid sequence identity to the SP ORF as used in Clynav.

### Example 2: The Plasmids

Plasmids according to the invention were prepared using procedures and materials that are well-known in molecular biology and are readily available. In short:

The eucaryotic expression plasmid pCI was purchased (Promega), and modified: the AmpR gene and its promoter were replaced by a fab gene and a promoter as described below.

Also the SP gene (from SAV3 isolate PD03.13, as described above) was inserted into the multiple cloning site of the pCI plasmid as an EcoRI-NotI fragment of almost 4 kb, in-between the hCMV IE1 gene promoter and the SV40 Late polyA-terminator signal. The final plasmid constructs were sequenced and verified.

2.1. Plasmid ‘pCI-SAV3 SP-Cat Prom-fabI’ (SEQ ID NO: 2)

The fabI gene (SEQ ID NO: 1) and the CatR gene promoter (GenBank acc. nr KX273378) were derived from published E. coli sequences, and were synthesized. Because the fabI gene was already derived from E. coli it did not require codon-optimisation. The CatR gene promoter and the fabI gene were then inserted into pCI, replacing the AmpR gene and -promoter. The resulting plasmid according to the invention: ‘pCI-SAV3 SP-Cat prom-fabI’, is as disclosed in SEQ ID NO:2.

2.2. Plasmid ‘pCI-SAV3 SP-Kan Prom-fabV2’ (SEQ ID NO: 3)

The variant fab gene: fabV2 was derived from published sequences from Vibrio bacteria, and was then codon-optimised for E. coli. The KanR gene promoter was used to drive this fabV2 gene, so as to be comparable to the use of this promoter in the Clynav product (plasmid pUK-SPDV-poly2 #1).

The construct with KanR gene promoter and fabV2 gene was inserted into a pCI plasmid, instead of its AmpR gene and -promoter. The resulting plasmid according to the invention: ‘pCI-SAV3 SP-Kan prom-fabV2’, is as disclosed in SEQ ID NO:3.

### 2.3. Plasmid Preparation

Both plasmids were amplified in E. coli DH5alpha cells, in standard bacterial culturing medium: Luria-Bertani broth, comprising 1 μM triclosan. Plasmids were purified using QIAgen isolation kits according to the manufacturer's instructions; this type of purification in principle provides sterile plasmid preparations.

The plasmid to be used in the various vaccination-challenge trials was taken up into water-for-injection, and the total DNA concentration of the plasmid was determined by measuring the OD 260/280 using the NanoDrop. Based on this measurement of total plasmid DNA concentration the amount of plasmid per animal dose required for use in the various vaccination-challenge experiments, was taken up into PBS at the volume to be administered per animal.

### Example 3: Materials and Methods for the Vaccination-Challenge Experiments in Fish

A number of vaccination-challenge experiments were performed in young salmon that were kept under standard conditions. The fish were vaccinated with one of the plasmids according to the invention, or were given a control- or a mock vaccination. Next the fish were challenge-infected with a virulent SAV strain, and monitored for the occurrence of symptoms of disease caused by the SAV infection. All these procedures are well-known in the field of the invention; the procedures common to the various experiments were as follows:

### 3.1. Animal Experimental Procedures

### Animals:

Atlantic salmon (Salmo salar), strain: Stofnfiskur, of mixed sex, at pre-smolt age, were used in all experiments. Average weight at vaccination was 30 grams (±2 grams, n=20) each. Only healthy animals were used. After placing, fish could acclimatize for 7 days before vaccination.

Before marking and vaccination the fish were starved for 36-48 hours, and were then anaesthetized using Finquel™ Vet. (MSD Animal Health). Marking of the fish in the different treatment-groups was by clipping of the adipose fin and/or of the maxillae. Groups to be analysed by PCR had 35 fish/group; groups for analysis of heart lesions had 20 fish/group.

### Housing:

The fish were held in tanks of about 500 litres, in fresh water: salinity: 0%, pH: 6.8-7.2, temperature 12° C.±2° C., and oxygen at about 75% (range: between 65% [i.e. 8 mg/ml] and 100%, by regulation of waterflow), with a lighting regime of 12 hours light and 12 hours dark. Fish biomass density was below 40 kg/m³. Fish were fed according to appetite, using standard commercial salmon feed pellets.

### Vaccination:

Vaccination for the test-, control-, and mock groups, were administered in a volume of 50 μl per animal, and by intra-muscular route into the side of the fish, using an insulin syringe and needle of 29 G×½.

Test vaccines were the various plasmids according to the invention, as described below.

Mock vaccine was saline (0.9% NaCl). The control vaccine was taken from a commercial batch of Clynav; this commercial product was found to contain 10.6 μg of total plasmid DNA per dose, based on A260/280 measurement. In some trials a full dose of Clynav was given (‘10 μg’), in some others a reduced amount was used.

### Challenge:

The challenge-infection was administered at 6 weeks post vaccination, by cohabitation with infected shedders, as this is the most effective- and most natural-way of SAV infection. The shedders were naïve fish that were given a challenge dose of 2.8 Log 10 TCID50/animal of a virulent, low-passage, SAV3 virus strain (MSD AH: PD03-012, Vik), in 200 μl, by intraperitoneal route, at the day of challenge.

The inoculated shedders were then placed into the holding tanks with the test animals, at an amount of 20% of the final total number of fish. Because this challenge strain differs from the SAV3 strain that provided the SAV SP gene inserted in the plasmids according to the invention, this qualifies as a heterologous challenge. When dead fish were observed, these were removed from the tank.

### Serum Sampling:

Blood samples were taken at 3 weeks post challenge (i.e. 9 weeks post vaccination) for determination in the fish's serum of challenge virus viraemia; this is the period of the highest virus loads in (unvaccinated) cohabitation-challenged fish.

Blood was collected from the caudal vein of anesthetized fish and was left overnight at 4° C. for clotting. Serum was separated from the blood cells by centrifugation for 5 min. at 4000×g and 4° C., and individual fish serum samples were labelled and stored frozen at −80° C. until use.

### Viraemia Assessment:

Vaccination-efficacy, by determining challenge-virus viremia, was determined at three weeks post challenge (wpc), in serum samples, by way of a real-time Reverse Transcription Polymerase Chain Reaction (real-time RT-PCR) assay, using primers specific for the SAV nsP1 gene RNA. This qRT-PCR is described in detail below. The results of each test sample in reduction of prevalence of viremia was expressed as relative percent protection (RPP) of the test group compared to the negative control group.

3.2. Real-Time qRT-PCR

Fish serum test samples from frozen storage were thawed, and 50 μl aliquots were taken. Positive- and negative serum control samples were treated in a similar way.

An internal control for the RNA isolation and the PCR was performed in parallel by spiking all samples prior to RNA extraction with inactivated equine influenza virus (EIV) H3N8 (1 μl/sample, corresponding to at least 10\hat{ }5 TCID50 of EIV), and by detecting the presence of the EIV HA H3 gene RNA, as quality control for the RNA extraction.

Consequently, the PCR determination of vaccination efficacy was thus based on the result of performing two separate one-step real-time RT-PCR assays on dual aliquots of the same sample of total RNA isolated from a test serum sample:
a PCR assay for the detection of SAV, using SAV nsP1-specific primers named: ‘nsP1-F’ and ‘nsP1-R’, and a Taqman™ MGB probe named: ‘nsP1 probe’ that was labelled with FAM reporter dye, all according to Andersen et al., 2007 (Arch. Vir., vol. 152, p. 1871-1883), to provide an amplicon of 107 bp; and in parallel:
a PCR assay for the detection of the EIV control that was spiked into the test serum samples, using EIV HA H3 specific primers named: ‘IVA-EI-H3 forward’ and ‘IVA-EI-H3 Reverse-alt’, and a Taqman MGB probe named: ‘IVA-EI-H3-MGB’, all according to Foord et al., 2009 (Vet. Microbiol., vol. 137, p. 1-9), except that the EIV MGB probe was labelled with a VIC reporter dye instead of FAM. This provides an amplicon of 57 bp.

Total RNA was extracted from the serum samples using the RNeasy® 96 Kit (Qiagen), and the PCR kit used was the ABgene/ThermoFisher scientific Verso™ 1-Step Q-RT-PCR Kit low ROX, both according to the manufacturer's instructions. The PCR samples, at final volume of 25 μl each, were in MicroAmp® Fast Optical 96-Well Reaction Plates (Applied Biosystems), and were run on an ABI PRISM® 7500 Fast Sequence Detection System (Applied Biosystems), which was connected to a PC running ABI SDS software version 1.5.1. The PCR protocol used was: 1 cycle of 30 min. at 50° C.; 1 cycle of 15 min. at 95° C.; and 40 cycles of [15 sec. at 95° C., followed by 1 min. at 60° C.]. For the Ct results, the 0.00 value baseline is set based on cycles 3-15. The Ct results were then exported to an Excel™ file for calculation of RPP values.

TABLE 3
Primers and probes used in the qRT-PCR

| SEQ ID NO: | Detects | Name | nt sequence (5′→3') |
|---|---|---|---|
| 4 | SAV | nsP1-F | ccggccctgaaccagtt |
| 5 | | nsP1-R | gtagccaagtgggagaaagct |
| 6 | | nsP1 probe | [FAM]-tcgaagtggtggccag-[MGB-NFQ] |
| 7 | EIV | IVA-EI-H3 forward | cagcgctttcagcaattgc |
| 8 | | IVA-EI-H3 Reverse-alt | gaycggagcgatgcatagtc |
| 9 | | IVA-EI-H3-MGB | [VIC]-cccatatgacatccc-[MGB-NFQ] |

### Assessment of PCR Results:

For the SAV assay, a cycle threshold (Ct) value at or below 35.00 was considered to be indicative for the presence of SAV challenge virus in a serum test sample, as above Ct=35 the assay deviated from linearity. Scores above that value indicated the serum sample was free of SAV.

To assess validity, the positive- and negative SAV control samples needed to score accordingly, and all samples needed to score positive for the detection of the spiked EIV (i.e. a Ct value of 32.0 or less). Further, in the group of mock-vaccinated fish, at least 60% of the individual samples needed to be positive for SAV, to demonstrate an effective take of the challenge infection.

The 60% cut-off value is a common minimum level of challenge-induced disease as described in the European Pharmacopoeia for potency trials of fish vaccines.

### Calculation of RPP:

The presence of SAV challenge virus in the test sera was evaluated categorically (+ or −) as a percentage of total, based on Ct value, whereby a Ct≤35.00 was PCR positive for SAV. Next, the relative difference between the vaccinated- and the mock-vaccinated (i.e. negative control) groups was calculated as the RPP

### 3.3. Heart Lesion Scoring

In some trials the effect was determined of vaccination with a plasmid according to the invention, on the reduction of SAV specific heart lesions. For this purpose 20 additional fish were added to the various vaccination- and control groups. These were vaccinated and challenged as the other animals, but were not used for blood sampling at 3 weeks post challenge, but were used to isolate their hearts at 5 wpc for histopathological analysis as follows:

The whole heart (including atrium and bulbus arteriosus) was removed, and stored in separate labelled tubes with formalin until use. Heart tissue samples were prepared by standard paraffin wax technique, and microscopy slides were prepared and stained. These were analysed and scored for SAV induced heart lesions on a semi-quantitative scoring scale of 0-4:
0 Normal appearance
1 Focal myocardial degeneration with or without (‘±’) inflammation (<7% of fibres affected)
2 Multifocal myocardial degeneration±inflammation (<15% of fibres affected)
3 Moderate multifocal myocardial degeneration±inflammation (<50% of fibres affected)
4 Severe diffuse myocardial degeneration±inflammation (>50% of fibres affected)

This is derived from McLoughlin et al., 2006 (Dis. of Aquat. Organ., vol. 72, p. 125-133; Table 1). Histological heart lesion scores of 2 or higher were considered indicative of SAV infection, and the percentage of fish having a score ≥2 was reported.

3.4. Combination with a W/O Vaccine

In some trials a group was included that received an additional vaccination with a traditional oil-adjuvated vaccine, to investigate if that would interact or interfere with the DNA vaccination. The adjuvated vaccine employed contained antigens from a variety of fish pathogens: inactivated IPN virus, and several inactivated bacteria. It was formulated with a mineral oil adjuvant into a water-in-oil emulsion.

This W/O vaccine was given at the same time as the DNA vaccine, except that it was administered intraperitoneally at 0.1 ml/animal.

### Example 4: Results and Conclusions of the Vaccination-Challenge Experiments in Fish

4.1. Trial 1: Use of a fabI Containing Plasmid

In this experiment three different doses of the fabI containing plasmid: pCI-SAV3 SP-Cat prom-fabI were tested, next to a full dose of the Clynav vaccine. Both relative protection based on PCR of sera at 3 wpc, and SAV specific heart lesion scores at 5 wpc were assessed.

TABLE 4
Results of vaccination-challenge Trial 1

| | dose | heart lesions | RPP |
|---|---|---|---|
| Vaccine | (μg) | (% positive) | (%) |
| pCl-SAV3 SP-Cat prom-fabl | 10 | 0 | 100 |
| | 5 | 0 | 100 |
| | 2 | 0 | 97 |
| Clynav | 10 | 0 | 100 |
| Saline | 0 | 78 | — |

In Table 4, ‘dose’ is the amount of total plasmid DNA in micrograms that was administered per fish. The percentages for ‘heart lesions’ refer to fish with a histopathological lesion score of 2 or higher.

Clynav vaccine at a full commercial dose provided complete protection; a mock vaccination with saline did not provide any protection from detection of challenge virus at 3 wpc.

The score of 97% for the vaccination with 2 μg of the plasmid pCI-SAV3 SP-Cat prom-fabI, is not considered to be significantly different from the 100% scores for the other amounts of the invention vaccine; this is also supported by the full protection against heart lesions that was induced by that 2 μg dose vaccine. Also, upon repeat of this experiment, see Example 3.2 below, similar results were obtained.

### Conclusions

In this trial it was shown for the first time that a DNA vaccine against SAV according to the invention was as effective as an existing commercial SAV DNA vaccine, even when used at much reduced amounts of plasmid DNA per dose of half, or even of ⅕^{th} of the amount used for an existing SAV DNA vaccine.

4.2. Trial 2: Use of fabI Plasmid in Combination with a W/O Vaccine

In this trial, the experiment of Trial 1 was repeated, and the potential effect of the simultaneous administration of a W/O vaccine was tested.

TABLE 5
Results of vaccination-challenge Trial 2

| | dose | RPP |
|---|---|---|
| Vaccine | (μg) | (%) |
| pCl-SAV3 SP-Cat prom-fabl | 8 | 100 |
| | 5 | 97 |
| | 2 | 100 |
| pCl-SAV3 SP-Cat prom-fabl + W/O vaccine | 5 | 100 |
| Clynav | 10 | 100 |
| Saline | 0 | — |

### Conclusions

In this animal experiment, similar results were obtained as found in Trial 1: the plasmid pCI-SAV3 SP-Cat prom-fabI is equally effective as DNA vaccine against SAV as a commercial product, even when given at a substantially reduced amount of plasmid DNA per dose.

Thus, as is evident from the results of Trials 1 and 2, even a dose of 2 μg per animal of total plasmid DNA of a plasmid according to the invention, can completely prevent the detection by means as sensitive as PCR, of SAV challenge virus in its serum, at 3 weeks after a challenge with a virulent SAV of a heterologous SAV3 strain.

Also, the simultaneous administration of a traditional W/O vaccine against other pathogens, did not have a significant negative effect on the level of SAV protection obtained, based on RPP.

4.3. Trial 3: Use of a fabV2 Plasmid at Further Reduced Dose

To investigate how far the amount of plasmid DNA in the vaccine according to the invention could be reduced, the plasmid: pCI-SAV3 SP-Kan prom-fabV2 according to the invention, and the Clynav DNA vaccine were tested at amounts of only 1 or 0.2 μg total plasmid DNA per animal dose.

TABLE 6
Results of vaccination-challenge Trial 3

| | dose | RPP |
|---|---|---|
| Vaccine | (μg) | (%) |
| pCl-SAV3 SP-Kan prom-fabV2 | 1 | 97 |
| | 0.2 | 66 |
| Clynav | 1 | 54 |
| | 0.2 | 11 |
| Saline | 0 | — |

### Conclusions

As is evident from the results in Table 6, a dose of only 1 μg of the plasmid pCI-SAV3 SP-Kan prom-fabV2, was still able to prevent detection of essentially all of the virulent heterologous SAV3 challenge virus at 3 wpc. Also, when the minimal level of RPP required is set at 55, 60% or even at 65%, a dose of only 0.2 μg of a plasmid according to the invention still qualifies as effective DNA vaccine against SAV.

At the same time however, the Clynav vaccine clearly does not meet that limit of the RPP level at 1 μg dose, and certainly not at 0.2 μg dose.

In this regard it is relevant to note that the pCI-SAV3 SP-Kan prom-fabV2 plasmid also employs the hCMV IE1 gene promoter to drive the SAV SP gene, and also uses the KanR gene promoter to drive the gene for phenotypic selection, both as are applied in the plasmid pUK-SPDV-poly2 #1 of Clynav. Consequently the main difference between these plasmids, the presence of a fab gene, is considered to be responsible for this much-improved efficacy of the SAV DNA vaccine according to the invention at (very) low amount of plasmid DNA/animal dose.

### 4.4. Trial 4: Test of Duration-of-Immunity and Cross-Protection

The experiment of Trial 2 was repeated to assess the duration-of-immunity (DOI) after DNA vaccination with different doses of a plasmid according to the invention: the plasmid pCI-SAV3 SP-Cat prom-fabI, against SAV3 challenge infection at 9 and at 12 months post vaccination (mpv). Also, the effect on DOI of a simultaneous immunisation with an oil-adjuvanted vaccine was tested at these time points.

Further, to assess the duration of cross-protection efficacy, one of the groups was challenged with SAV2 at 12 mpv.

Most experimental conditions were the same as for the previous trials: group size was 35; 50 μl volume was given i.m. for the DNA vaccine, and for the control (saline); for the multivalent W/O vaccine 100 μl was given i.p.; for the SAV3 challenged groups, blood was collected at 3 weeks pc. Detection of challenge virus prevalence in serum was by SAV specific RT qPCR, using the conserved nsP1 primers.

A difference was that the fish had been marked using passive integrated transponder (PIT) tags, to assure proper marking throughout the duration of the trial. The challenge with a virulent mSAV2 (strain: SAV2-VI, passage 1) was given by direct i.m. inoculation with 50 μl of mSAV2 virus dilution. Blood was collected at 10 dpc.

TABLE 7
Results of vaccination-challenge Trial 4

| | | RPP (%) against challenge: | | |
|---|---|---|---|---|
| | dose | SAV3 | | mSAV2 |
| Vaccine | (μg) | 9 mpv | 12 mpv | 12 mpv |
| pCl-SAV3 SP-Cat prom-fabl | 8 | 96 | 100 | 100 |
| | 5 | 100 | 100 | 94 |
| | 2 | 96 | 100 | 69 |
| pCl-SAV3 SP-Cat prom-fabl + | 5 | 96 | 100 | 97 |
| W/O vaccine | | | | |
| Saline | 0 | — | — | — |

In the Control groups the challenge virus prevalence detected in serum was: 80% (SAV3, 9 mpv); 69% (SAV3, 12 mpv); and 100% (mSAV2, 12 mpv). This indicates that all challenges were well above the required minimal severity, and that the mSAV2 challenge was even very heavy.

### Conclusions

Trial 4 again showed surprisingly good protection from the plasmid DNA vaccine according to the invention, even at a low dose, and even at 9 and at 12 months post vaccination. All doses of 8, 5 and 2 μg were fully protective over the whole duration tested. The simultaneous administration of an oil-adjuvated vaccine did not have an effect on protection or DOI.

With regard to cross-protection against another SAV subtype, the doses of 8 and 5 μg were fully protective against the severe mSAV2 challenge infection, at 12 months post vaccination, and even when administered in combination with a multivalent oil-adjuvanted vaccine. The 2 μg dose vaccine was a little less cross-protective at 12 mpv, but still gave an acceptable level of (cross-) protection.

Consequently, the overall conclusion can be that the DNA vaccines according to the invention are at least as effective as an existing commercial DNA vaccine for salmonids against SAV. They are effective at a lower amount of DNA per dose, they provide immunity for an extended period, and they can be administered in simultaneous use with an oil-adjuvanted vaccine. Further they are effective against several SAV subtypes.

1. A plasmid comprising a gene encoding an antigen from salmonid alphavirus (SAV) and a eucaryotic promoter operably linked to said gene, wherein said plasmid also comprises a fab gene.

2. The plasmid according to claim 1, wherein the SAV is selected from SAV1, SAV2, and SAV3.

3. The plasmid according to claim 1, wherein the antigen from SAV is the structural polyprotein (SP) of SAV.

4. The plasmid according to claim 1, wherein the eucaryotic promoter is an hCMV IE1 gene promoter.

5. The plasmid according to claim 1, wherein the fab gene is fabI or is a mutant, homolog, or variant of fabI.

6. The plasmid according claim 5, wherein the fab gene is selected from: fabI, fabK, fabL, fab V, mfabI, fabI2, fabL2, and fab V2.

7. The plasmid according to claim 1, wherein the plasmid is based on a pCI plasmid.

8. The plasmid according to claim 1, wherein the plasmid has a nucleotide sequence that has at least 90% nucleotide sequence identity to the full length of SEQ ID NO: 2, or of SEQ ID NO: 3.

9. Host cell comprising the plasmid according to claim 1.

10. (canceled)

11. A DNA vaccine comprising the plasmid according to claim 1 and a pharmaceutically acceptable carrier.

12. (canceled)

13. A method for the protection of fish against infection or a disease caused by SAV, the method comprising administering to said fish the DNA vaccine according to claim 11.

14. The method of claim 13, wherein the fish is a Salmonid.

15. (canceled)

16. A method for the preparation of the DNA vaccine according to claim 11, comprising the step of admixing a plasmid comprising a gene encoding an antigen from salmonid alphavirus (SAV) and a eucaryotic promoter operably linked to said gene, wherein said plasmid also comprises a fab gene and a pharmaceutically acceptable carrier.

17. A method to increase the efficacy of the plasmid in a SAV DNA vaccine, the method comprising the step of inserting a fab gene into said plasmid.

18. The method of claim 13, wherein the fish are administered the DNA vaccine at a dose of less than 5 ug.

19. The method of claim 18, wherein the fab gene is fabI or is a mutant, homolog, or variant of fabI.

20. The method of claim 18, wherein the fab gene is selected from: fabI, fabK, fabL, fab V, mfabI, fabI2, fabL2, and fab V2.

21. The method of claim 18, wherein the fab gene is selected from the group consisting of:
a. a fab gene has a nucleotide sequence that is at least 97% sequence identity to the full length of the nucleotide sequence of SEQ ID NO: 1;
b. a fabV2 gene that is the region from nucleotide 6329 up to and including nucleotide 7534 from SEQ ID NO: 3, or a nucleotide sequence having at least 97% sequence identity to the region from nucleotide 6329 up to and including nucleotide 7534 from SEQ ID NO: 3, and
c. a fabI gene that is the region from nucleotide 6294 up to and including nucleotide 7082 from SEQ ID NO: 2, or a nucleotide sequence having at least 97% sequence identity to the region from nucleotide 6294 up to and including nucleotide 7082 from SEQ ID NO: 2.
